(19) United States
(12) Patent Application Publication
(10) Pub. No.: US 2004/0030297 A1
(43) Pub. Date: Feb. 12, 2004

(54) Device for guiding a drive in an injection device

(76) Inventor: Thomas Hegewald, Baiersdorf (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/438,225

(22) Filed: May 14, 2003

(30) Foreign Application Priority Data
May 15, 2002 (DE) 10221783.1-44

(51) Int. Cl. A61M 5/00

(52) U.S. Cl. 604/207

(57) ABSTRACT

The present invention relates to a device for guiding a rod-shaped output member (401) of an injection device with an exchangeable cartridge containing injection liquid, the rod-shaped output member (401) having grooves (402) extending in lines in the axial direction on the outer circumference and, between the grooves (402), lines (403) with a structured surface, a guide element made up of at least two parts being provided, both parts of which are of annular shape and each have grooves (301, 302) extending in lines in the axial direction on their inner circumference and, between the grooves, lines (303, 304) with a structured surface, the two parts of the guide element being turned relative to one another in a working position in such a way that the grooves of the one part are continued in the axial direction by the structured surface of the other part.

The invention relates to a device for guiding a rod-shaped output member of an injection device.

### BACKGROUND OF THE INVENTION

The Applicant markets an injection appliance under the name OptiPen® for insulin injection. This injection appliance contains a cartridge with insulin. This cartridge is exchangeable. In the injection appliance, the insulin is dispensed from the cartridge by means of a plunger reducing the volume of the cartridge. On the other side of the cartridge, a membrane forming part of the surface of the cartridge is pierced by the injection needle. Upon reduction of the volume, the insulin is thus dispensed through the injection needle. When the cartridge is to be changed, the plunger is located in the farthest forward position because the cartridge situated in the appliance has been at least almost completely emptied. However, in order to be able to insert a new, full cartridge, the plunger has to be moved back to the rearmost position. The cartridge is secured in the injection appliance by means of a screw thread.

### SUMMARY OF THE INVENTION

The plunger rod of the injection appliance, OptiPen®, corresponds to the rod-shaped output member of the present invention. To be able to move the plunger rod back to the rearmost position, the rod-shaped output member has grooves extending in lines in the axial direction on the outer circumference and, between the grooves are situated lines with a structured surface, e.g., a thread, lead scew, or a lead screw having two or more flats. In the illustrative embodiment described herein, this structured surface is a thread.

Furthermore, a guide element made up of at least two parts is provided, both parts of which are of annular shape and each have grooves extending in lines in the axial direction on their inner circumference and, between the grooves are situated lines with a structured surface. Here, the structured surface corresponds to an internal thread.

The problem on which the present invention is based is that of simplifying the handling of the injection device.

According to the invention, this is achieved by the fact that the cartridge can be secured in the injection device by means of a bayonet catch (FIG. 1, 101, 102), the rotation for opening the bayonet catch corresponding in terms of direction of rotation and angle of rotation to the turning of the second part of the guide element relative to the first part for bringing the guide element from the working position to the unlocked position, and, when the bayonet catch is opened, the locking of the angle position of the two parts of the guide element in the working position is canceled and the second part is turned relative to the first part.

The present invention thus affords the advantage that separate manual setting of the unlocked position can be eliminated when changing the cartridge. The fact that, according to the present invention, a bayonet catch is provided for securing the cartridge affords the advantage that when removing the old cartridge, and also when inserting the new cartridge, turning takes place about a defined angle. This angle can advantageously be so dimensioned that said angle corresponds to the turning of the two parts relative to one another from the working position to the unlocked position.

### BRIEF DESCRIPTION OF THE FIGURES

An illustrative embodiment of the invention is shown in the drawing, in which:

FIG. 1 shows the main parts of an injection device for the present invention,

FIG. 2 shows the rotary disk 4 in a detailed representation from a number of views,

FIG. 3 shows a representation of the second part 3 of the guide element, and

FIG. 4 shows an exploded view of the injection device with all the individual parts.

### PREFERRED EMBODIMENTS

An embodiment of the invention provides a device according to the present invention, wherein in a working position, the two parts of the guide element are turned relative to one another in such a way that the grooves of the one part are continued in the axial direction by the structured surface of the other part. This means that, in the working position, at least one of the two parts is always in engagement via its internal thread with the thread of the rod-shaped output member. In the working position, the two parts of the guide element are locked securely against rotating relative to one another.

An embodiment of the invention provides a device according to the present invention, wherein the rod-shaped output member is mounted securely against rotation in the injection device.

An embodiment of the invention provides a device according to the present invention, wherein, before the dose quantity of an injection is set, the plunger is situated at a defined spacing from the cartridge. When an injection is triggered, the plunger is moved by a defined distance toward the cartridge. The spacing is dimensioned in such a way that the spacing corresponds exactly to the defined distance. Thus, if no dose is set, an insulin quantity of “0” is injected, because the defined distance corresponds exactly to the defined spacing and the plunger then bears precisely on the cartridge without having reduced the latter's volume.

An embodiment of the invention provides a rotatable setting element on the injection device, with which the dose quantity can be set. This is done, before the injection is triggered, by the plunger being moved toward the cartridge by a distance corresponding to the dose quantity. With the rotatable setting element, the already described guide element comprising the two parts is turned. As a result of the turning of the guide element and the rotationally fixed mounting of the rod-shaped output member, the rod-shaped output member is moved forward corresponding to the pitch of the thread.

An embodiment of the invention provides a device wherein, in the working position, the guide element can be turned relative to the rod-shaped output member by integral multiples of a defined angle. At the start of the turning, a first of the two parts is situated relative to the rod-shaped output element in a position in which the structured surface of the inner circumference of the one part is located opposite the grooves of the rod-shaped output member, and a second of the two parts is located relative to the rod-shaped output element in a position in which the structured surface of the inner circumference of the one part is located opposite the structured surface of the rod-shaped output member. This guarantees that a defined relative angle position is obtained between the two parts of the guide element and the rod-shaped output member.

An embodiment of the invention provides a device wherein the angle position of the two parts of the guide element relative to one another is locked in the working position. This therefore ensures that in the working position at least one of the threads of the two parts is always in engagement with the thread of the rod-shaped output member.

An embodiment of the invention provides a device wherein the the two parts of the guide element can be brought into an unlocked position. By means of a manual actuation by the user, the locking of the two parts relative to one another in the working position is canceled. Then, by means of an actuation by the user, the second part is turned relative to the first part so that the grooves of the one part are continued in the axial direction by the grooves of the other part. Based on the above-described definitions of the angle position of the first part relative to the rod-shaped output member, the structured surface of the two parts lies opposite the grooves of the rod-shaped output member in this unlocked position. The engagement of the threads of the two parts of the guide element with the thread of the rod-shaped output member is thus released. In this unlocked position, the rod-shaped output member is thus freely movable in the axial direction. By a suitable inclination of the injection device, it can thus fall back into the starting position.

An embodiment of the invention provides a device wherein the two parts of the guide element can be locked, for example, by means of the two parts having grooves extending along their outer circumference at corresponding angle positions, into which grooves rod-shaped elements can be pushed for locking. By removing the rod-shaped elements, the locking can be released.

An embodiment according to the invention is a device for guiding a rod-shaped output member (401) of an injection device with an exchangeable cartridge containing injection liquid, the rod-shaped output member (401) having grooves (402) extending in lines in the axial direction on the outer circumference and, between the grooves (402), lines (403) with a structured surface, a guide element made up of at least two parts being provided, both parts of which are of annular shape and each have grooves (301, 302) extending in lines in the axial direction on their inner circumference and, between the grooves (301, 302), lines (303, 304) with a structured surface, the two parts of the guide element being turned relative to one another in a working position in such a way that the grooves of the one part are continued in the axial direction by the structured surface of the other part, and, in the working position, the guide element can be turned relative to the rod-shaped output member (401) by integral multiples of a defined angle, and, at the start of the turning, a first of the two parts is situated relative to the rod-shaped output element (401) in a position in which the structured surface of the inner circumference of the one part is located opposite the grooves (402) of the rod-shaped output member (401), and a second (3) of the two parts is located relative to the rod-shaped output element (401) in a position in which the structured surface (303, 304) of the inner circumference of the second part is located opposite the structured surface (403) of the rod-shaped output member (401), and the angle position of the two parts of the guide element is locked in the working position, and the two parts can be brought into an unlocked position by means of the second part (3) being rotatable relative to the first part in such a way that the grooves of the one part are continued in the axial direction by the grooves of the other part, and the structured surface of the two parts lies opposite the grooves (402) of the rod-shaped output member (401) in this unlocked position, wherein the cartridge can be secured in the injection device by means of a bayonet catch, the rotation for opening the bayonet catch corresponding in terms of direction of rotation and angle of rotation to the turning of the second part (3) of the guide element relative to the first part for bringing the guide element from the working position to the unlocked position, and, when the bayonet catch is opened, the locking of the angle position of the two parts of the guide element in the working position is canceled and the second part is turned relative to the first part (4).

Another embodiment according to the invention is a device for guiding a rod-shaped output member (401) of an injection device with an exchangeable cartridge containing injection liquid, the rod-shaped output member (401) having grooves (402) extending in lines in the axial direction on the outer circumference and, between the grooves (402), lines (403) with a structured surface, a guide element made up of at least two parts being provided, both parts of which are of annular shape and each have grooves (301, 302) extending in lines in the axial direction on their inner circumference and, between the grooves (301, 302), lines (303, 304) with a structured surface, the two parts of the guide element being turned relative to one another in a working position in such a way that the grooves of the one part are continued in the axial direction by the structured surface of the other part, and, in the working position, the guide element can be turned relative to the rod-shaped output member by integral multiples of a defined angle, the angle position of the two parts of the guide element being locked in the working position, and the two parts can be brought into an unlocked position by means of the second part (3) being rotatable relative to the first part in such a way that the grooves of the one part are continued in the axial direction by the grooves of the other part, and the structured surface of the two parts lies opposite the grooves (402) of the rod-shaped output member (401) in this unlocked position, wherein the cartridge can be secured in the injection device by means of a bayonet catch, the rotation for opening the bayonet catch corresponding in terms of direction of rotation and angle of rotation to the turning of the second part of the guide element relative to the first part for bringing the guide element from the working position to the unlocked position, and, when the bayonet catch is opened, the locking of the angle position of the two parts of the guide element in the working position is canceled and the second part is turned relative to the first part (4).

Another embodiment of the present invention is the said device of the invention, wherein the two parts of the guide element can be fixed, in terms of their angle position, in the unlocked position.

Another embodiment of the present invention is the said device of the invention, wherein the fixing and/or locking of the two parts of the guide element is released by the travel of the bayonet catch upon opening/locking of the bayonet catch.

Another embodiment of the present invention is the said device of the invention, wherein by means of the travel of the bayonet catch upon opening/closure of the bayonet catch, a carrier element (4) connected to the bayonet catch, at least during the latter's rotation movement, comes into engagement (201, 305, 306) on the second part of the guide element during the rotation movement of the bayonet catch and, in this way, this second part of the guide element is rotated along with it.

It is appreciated that certain features of the invention, which are, for clarity, described in the context of separate embodiments, may also be provided in combination in a single embodiment. Also, various features of the invention which are, for brevity, described in the context of a single embodiment, may also be provided separately or in any suitable sub-combination.

In the configuration of the device in the present invention, it is not necessary for the two parts of the guide element at the start of an injection procedure to have a correspondingly defined angle position with respect to the rod-shaped output member. In order to reach the unlocked position, the second part can in this case also be turned relative to the first part about the corresponding angle. By further turning, it can then for example be achieved that the second part then entrains the first part until the correspondingly defined angle position is reached in which the rod-shaped output member is freely movable in the axial direction. This can be obtained, for example, by corresponding limit stops.

In the configuration of the device in the present invention, the two parts of the guide element can be fixed, in terms of their angle position, in the unlocked position.

This affords the advantage that, also when reinserting the new cartridge, the two parts are in a defined angle position with respect to one another, so that a defined operating state can once again be obtained.

This fixing can be achieved, for example, by the two parts again having grooves extending along their outer circumference at corresponding angle positions, into which grooves rod-shaped elements can be pushed for fixing. By removing the rod-shaped elements, the fixing can be released. These can be the same grooves and the same rod-shaped elements with which the locking in the working position can also be obtained.

In the configuration of the device in the present invention, the fixing and/or locking of the two parts of the guide element is released by the travel of the bayonet catch upon opening/closure of the bayonet catch.

By means of said travel, the rod-shaped elements can for example be pushed out at least from the grooves of one of the two parts. In this way, the two parts can then be turned relative to one another. Using the travel of the bayonet catch upon opening and also upon closure in order to release the fixing/locking affords the advantage that, at the start of the turning procedure, the fixing/locking is released so that turning is then made possible.

By the resetting of the travel at the end of the turning movement, it can at the same time be ensured that, in this end position, the fixing/locking once again automatically sets in.

Thus, with little outlay in construction terms, the rotation movement of the two parts relative to one another can advantageously be made possible by using the bayonet catch.

In the configuration of the device in the present invention, by means of the travel of the bayonet catch upon opening/closure of the bayonet catch, a carrier element connected to the bayonet catch, at least during the latter's rotation movement, comes into engagement on the second part of the guide element during the rotation movement of the bayonet catch. In this way, this second part of the guide element is rotated along with it.

The fact that this carrier element once again comes into engagement through the travel movement means that, upon resetting of the travel movement, this engagement is once again released, so that the guide element as a whole, with its two parts locked against rotation relative to one another, can be correspondingly rotated in the injection device.

FIG. 1 shows the main parts of an injection device. A main housing 2 can be seen on which a cartridge case 1 can be secured by means of a bayonet catch. The cartridge case serves here to receive the cartridge containing the insulin. It will be seen that the cartridge case has two notches 101 which engage with corresponding lugs on the rotary disk 4. When the cartridge case 1 is turned to secure it by means of the bayonet catch, the rotary disk 4 is therefore also turned along with it.

The second part 3 of the guide element can also be seen; the first part of the latter is not shown.

The bayonet catch can for example be formed by the projection on the cartridge case 1 and an insert part 102 which can be inserted into the main housing 2.

FIG. 2 shows the rotary disk 4 in a detailed representation from more than one view. It will be seen here that the rotary disk 4 has two projections 201 which engage in corresponding grooves of the second part 3 when the rotary disk 4 is moved in the direction of the second part 3. This movement can occur by means of the fact that, at the start of the turning of the bayonet catch, the travel movement takes place before the catch is opened/closed by the rotation movement of the bayonet catch. By means of the engagement of the projections 201 in the grooves of the second part 3 of the guide element, the second part 3 is entrained with the rotation movement of the bayonet catch. The rotary disk then therefore acts as a carrier element for the second part 3 of the guide element.

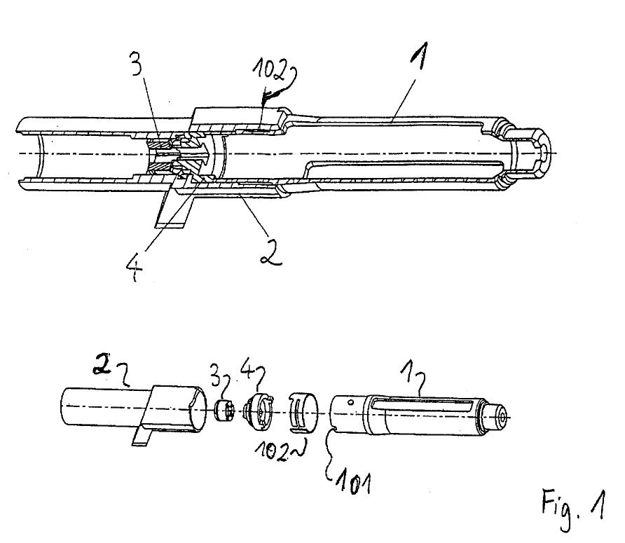

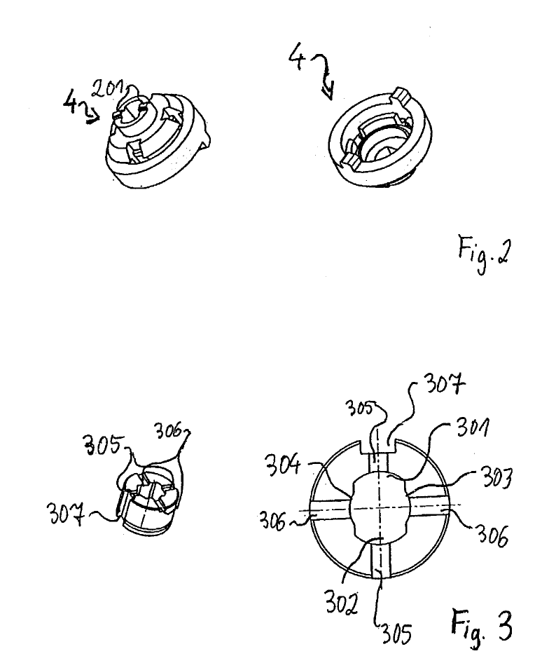

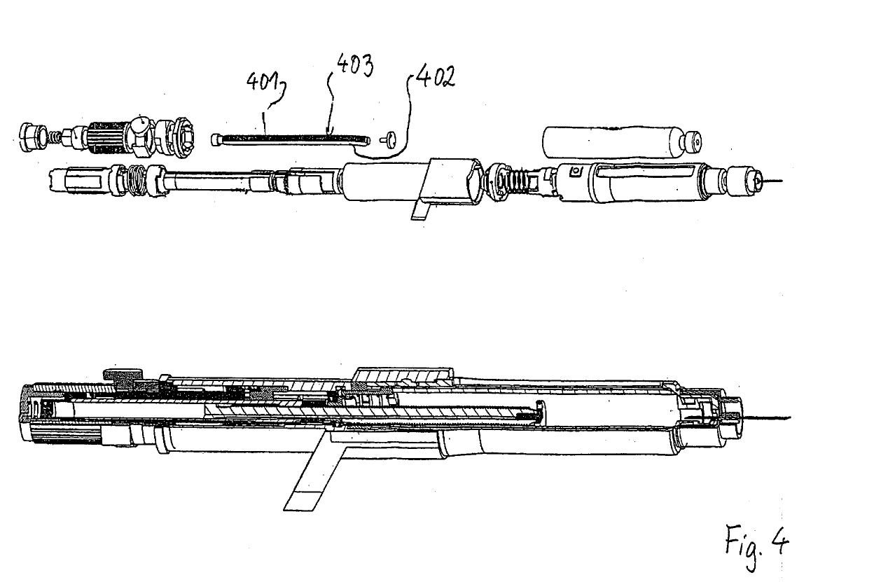

FIG. 3 shows a representation of the second part 3 of the guide element. It will be seen that this second part is annular in shape and has two grooves 301, 302 on its inner circumference. It will also be seen that between these grooves there are areas 303, 304 at which the second part has an internal thread. The rod-shaped output member shown in connection with FIG. 4 also has, along its outer circumference, areas which are milled out and areas on which there is a thread. When, in the relation between the second part and the rod-shaped output member, the grooves 301, 302 lie exactly opposite the areas of the rod-shaped output member at which the thread is located, the rod-shaped output member is freely movable in the axial direction relative to the second part.

In view of the initially described coordination of the angle positions of the first part and of the second part, this then also applies, in the unlocked position, to the first part.

In the working position, by contrast, at least one of the areas of the first part or second part at which there is a thread engages in a part of the rod-shaped output member at which there is a thread. The rod-shaped output member is then not freely movable in the axial direction but is instead guided in the axial direction by a corresponding rotation of the guide element.

The representation in FIG. 3 also shows the grooves 305, 306 of the second part 3 into which the projections 201 of the rotary disk 4 can engage.

Furthermore a groove 307 on the outer face of the second part 3 can also be seen. By means, for example, of a rod-shaped element which is pushed into this groove 307 and which at the same time has a rotationally fixed connection to the first part, it is possible to obtain a rotationally secured orientation of the second part 3 relative to the first part. For this purpose, the first part can also have a corresponding groove for example, through which the rod-shaped element is likewise pushed.

FIG. 4 shows an exploded view of the injection device with all its individual parts. The rod-shaped output member 401 can also be seen here. This rod-shaped output member has milled-out grooves 402 extending in the longitudinal direction on its outer circumference, and lines 403 on which a thread is located.

The present invention may be embodied in other specific forms without departing from the spirit or essential attributes thereof.

1. A device for guiding a rod-shaped output member (401) of an injection device with an exchangeable cartridge containing injection liquid, wherein the rod-shaped output member (401) has grooves (402) extending in the axial direction on the outer circumference and, between the grooves (402), are situated lines (403) with a structured surface; said device comprising a guide element made up of at least two parts, wherein both parts are
(i) annular in shape; and
(ii) have grooves (301, 302) extending in lines in the axial direction on their inner circumference;
wherein between the grooves (301, 302) are situated lines (303, 304) with a structured surface;
in a working position, the two parts of the guide element can be:
(i) turned relative to one another in such a way that the grooves of the one part are continued in the axial direction by the structured surface of the other part; and
(ii) turned relative to the rod-shaped output member (401) by integral multiples of a defined angle;
wherein, in the working position, the angle of the two parts of the guide element can be locked; and the two parts of the guide element can be brought into an unlocked position by means of:
(i) the second part (3) of the guide element being rotatable relative to the first part of the guide element in such a way that the grooves of the first part of the guide element are continued in the axial direction by the grooves of the second part of the guide element; and
(ii) the structured surface of the two parts lies opposite the grooves (402) of the rod-shaped output member (401) in this unlocked position; and
wherein the cartridge can be secured in the injection device by means of a bayonet catch, the the bayonet catch can be opened by rotating the first part (4) of the guide element in a direction and angle relative to the turning of the second part (3) of the guide element, so as to bring the guide element from the working position to the unlocked position; and
when the bayonet catch is opened, the locking of the angle position of the two parts of the guide element in the working position is canceled and the second part (3) is turned relative to the first part (4).

2. A device according to claim 1, wherein at the start of the turning of the guide element relative to the rod shaped output member, a first of the two parts of the guide element is situated relative to the rod-shaped output element (401) in a position wherein the structured surface of the inner circumference of the first part of the guide element is located opposite the grooves (402) of the rod-shaped output member (401), and the second (3) part of the guide element is located relative to the rod-shaped output element (401) in a position wherein the structured surface (303, 304) of the inner circumference of the second part is located opposite the structured surface (403) of the rod-shaped output member (401).

3. The device as claimed in claim 1, wherein the two parts of the guide element can be fixed, in terms of their angle position, in the unlocked position.

4. The device as claimed in claim 1, wherein the fixing and/or locking of the two parts of the guide element is released by the travel of the bayonet catch upon opening/locking of the bayonet catch.

5. The device as claimed in claim 1, wherein, by means of the travel of the bayonet catch upon opening/closure of the bayonet catch, a carrier element (4) connected to the bayonet catch, at least during the latter's rotation movement, comes into engagement (201, 305, 306) on the second part of the guide element during the rotation movement of the bayonet catch and, in this way, this second part of the guide element is rotated along with it.
